(19) United States
(12) Patent Application Publication
(10) Pub. No.: US 2026/0120839 A1
(43) Pub. Date: Apr. 30, 2026

(54) METHOD AND APPARATUS FOR PROVIDING ARTIFICIAL INTELLIGENCE-BASED OFF-LABEL INDICATIONS

(71) Applicant: SELTA SQUARE CO., LTD., Seoul (KR)

(72) Inventors: Min Kyung SHIN, Hwaseong-si (KR); Min Su LEE, Seoul (KR); Sol Ji YANG, Seoul (KR); Sergey DENISOV, Seoul (KR)

(21) Appl. No.: 19/373,600

(22) Filed: Oct. 29, 2025

(30) Foreign Application Priority Data
Oct. 31, 2024 (KR) 10-2024-0152266

(51) Int. Cl. G16H 20/10; G06N 3/0475; G16H 50/70

(52) U.S. Cl. CPC G16H 20/10; G06N 3/0475; G16H 50/70

(57) ABSTRACT

The present disclosure relates to a method and apparatus for providing an off-label indication in an off-label indication provision system, and the method for providing an off-label indication may include collecting real world data from health care big data, collecting drug-related information, converting the drug-related information and the real world data to international standard terms, generating relationship information by analyzing an indication relevant to an active ingredient based on the drug-related information and the real world data that are converted to the international standard terms, filtering an indication with low relevance to the active ingredient from the relationship information, and providing an off-label indication based on the filtered relationship information.

### CROSS-REFERENCE TO RELATED APPLICATION

This application claims priority to and the benefit of Korean Patent Application No. 10-2024-0152266, filed on Oct. 31, 2024, the disclosure of which is incorporated herein by reference in its entirety.

### BACKGROUND

### Field of the Invention

The present disclosure relates to a method and apparatus for providing an artificial intelligence (AI)-based off-label indication.

### Discussion of Related Art

Currently, new drug development is a costly and time-consuming process. Specifically, the success rate of new drug development is only about 2%, and the average development period is over 10 years. Off-label use refers to the use of a drug for an indication or at a dosage other than an indication for which the drug is approved. Since such off-label use is based on the judgment of medical professionals in actual clinical practice, it may reflect a variety of treatment situations. Therefore, there is a growing need to evaluate the potential for expanding the indications of existing drugs by identifying indications that are already used in clinical practice but have not yet been approved.

### SUMMARY

The present disclosure is directed to providing a method and apparatus for providing an off-label indication by using an artificial intelligence.

The present disclosure is directed to providing a method and apparatus for providing an off-label indication by analyzing real world data.

The present disclosure is directed to providing a method and apparatus for utilizing an artificial intelligence to automatically detect and analyze an off-label use case in real world data of a medical organization.

The present disclosure is directed to providing a method and apparatus for providing guidance on off-label use to utilize real world data as supporting evidence for drug repositioning.

The technical problems solved by the present disclosure are not limited to the above technical problems and other technical problems which are not described herein will become apparent to those skilled in the art from the following description.

According to an embodiment of the present disclosure, a method of providing an off-label indication in an off-label indication provision system includes collecting real world data from health care big data, collecting drug-related information, converting the drug-related information and the real world data to international standard terms, generating relationship information by analyzing an indication relevant to an active ingredient based on the drug-related information and the real world data that are converted to the international standard terms, filtering an indication with low relevance to the active ingredient from the relationship information, and providing an off-label indication based on the filtered relationship information.

According to an embodiment of the present disclosure, the collecting of the drug-related information may further include receiving drug information from a user, collecting drug approval information based on the drug information, and extracting an indication based on the drug approval information.

According to an embodiment of the present disclosure, the indication may be extracted based on a large language model (LLM).

According to an embodiment of the present disclosure, the drug approval information may be collected based on an active ingredient approval information application programming interface (API) that is provided by the Ministry of Food and Drug Safety.

According to an embodiment of the present disclosure, the filtering may be performed based on association rules data mining.

According to an embodiment of the present disclosure, the association rules data mining may be performed further based on the real world data.

According to an embodiment of the present disclosure, the association rules data mining may be performed based on at least one of support, confidence, or lift.

According to an embodiment of the present disclosure, the method may further include automatically searching for literature relevant to the drug-related information and verifying the filtered relationship information based on the searched literature, and the provision of the off-label indication may be performed based on the verification.

According to an embodiment of the present disclosure, an apparatus of providing an off-label indication in an off-label indication provision system includes a storage unit that stores necessary information for operating the apparatus and a processor coupled to the storage unit, and the processor is configured to collect real world data from health care big data, to collect drug-related information, to convert the drug-related information and the real world data to international standard terms, to generate relationship information by analyzing an indication relevant to an active ingredient based on the drug-related information and the real world data that are converted to the international standard terms, to filter an indication with low relevance to the active ingredient from the relationship information, and to provide an off-label indication based on the filtered relationship information.

According to an embodiment of the present disclosure, the processor may be further configured to receive drug information from a user, to collect drug approval information based on the drug information, and to extract an indication based on the drug approval information.

According to an embodiment of the present disclosure, the processor may be further configured to automatically search for literature relevant to the drug-related information and to verify the filtered relationship information based on the searched literature, and the provision of the off-label indication may be performed based on the verification.

According to the present disclosure, an artificial intelligence can be utilized to provide an off-label indication.

According to the present disclosure, real world data can be analyzed to provide an off-label indication.

According to the present disclosure, an artificial intelligence can be utilized to automatically detect an off-label use case in real world data of a medical organization.

According to the present disclosure, an artificial intelligence can be utilized to automatically analyze an off-label use case in real world data of a medical organization.

According to the present disclosure, guidance on off-label use can be provided to utilized real world data as supporting evidence for drug repositioning.

The effects obtainable from the present disclosure are not limited to the above-mentioned effects, and other effects not mentioned herein will be clearly understood by those skilled in the art through the following descriptions.

### BRIEF DESCRIPTION OF THE DRAWINGS

FIG. 1 illustrates a structure of a system that provides an off-label indication according to an embodiment of the present disclosure.

FIG. 2 illustrates a structure of an apparatus according to an embodiment of the present disclosure.

FIG. 3 illustrates a structure of an artificial neural network applicable to a system according to an embodiment of the present disclosure.

FIG. 4 illustrates a structure of an off-label indication provision apparatus according to an embodiment of the present disclosure.

FIG. 5 illustrates a graphDB showing a relationship between an active ingredient and an indication according to an embodiment of the present disclosure.

FIG. 6 illustrates a graph visualizing a relationship between an active ingredient and an indication according to an embodiment of the present disclosure.

FIG. 7 is a flowchart of a method of providing an off-label indication according to an embodiment of the present disclosure.

### DETAILED DESCRIPTION

Hereinafter, embodiments of the present disclosure will be described in detail with reference to the accompanying drawings such that those skilled in the art may easily implement the embodiments. However, the present disclosure may be implemented in different forms and is not limited to the embodiments described herein.

In describing the embodiments of the present disclosure, a detailed description of known functions and configurations will be omitted when it may obscure the subject matter of the present disclosure. In addition, in the drawings, a portion that is irrelevant to the description of the present disclosure is omitted, and similar reference numerals refer to similar portions.

The present disclosure proposes a technology of providing an off-label indication based on an artificial intelligence. Particularly, the present disclosure is directed to discovering a new use of an existing drug and expanding the indication of an approved drug, thereby significantly reducing the period, cost, and risk of new drug development. There may be diverse approaches to drug repurposing and drug repositioning. The present disclosure proposes a method of utilizing an artificial intelligence and analysis of real world data. Here, the real world data may be data that is collected not from a restricted situation such as a clinical test but from an actual medical environment. As an example, real world data according to the present disclosure may be big data that is provided by a health insurance review & assessment service (HIRA). In the present disclosure, real world data may be analyzed to derive information on an actual medical environment. Hereinafter, a system of providing such an off-label indication will be described in detail. In the present disclosure, an indication may be expressed by the term “medical indication” or any other terms having the same meaning.

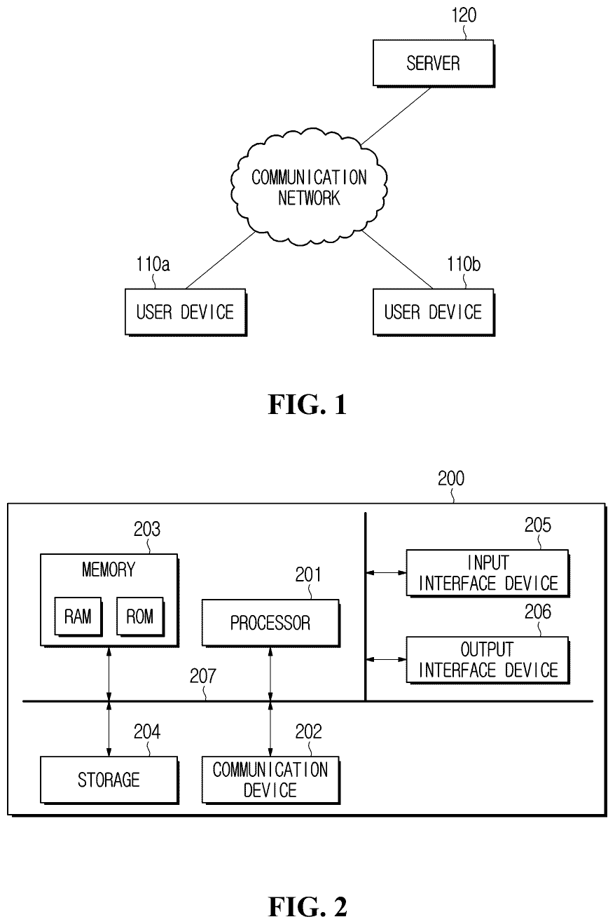

FIG. 1 illustrates a structure of a system that provides an off-label indication according to an embodiment of the present disclosure. Referring to FIG. 1, the system includes a user device 110a, a user device 110b, and a server 120 that are connected to a communication network. FIG. 1 illustrates the two user devices 110a and 110b, but three or more user devices may be present.

The user device 110a and the user device 110b may be used by users (e.g., a doctor or a professional) who needs an off-label indication through a platform according to an embodiment of the present disclosure. Here, the platform may refer to an operating system that constitutes the system for providing an off-label use indication according to the present disclosure. The user devices 110a and 110b may obtain input data (e.g., a fingerprint, a query, a URL, an email, a user's input, an electronic document, or real world data), transmit the input data to the server 120 via the communication network, and interact with the server 120. Each of the user devices 110a and 110b may include a communication unit for communication, a storage unit storing data and a program, a display unit for displaying information, an input unit for a user's input, and a processor for control. For example, each of the user devices 110a and 110b may be a general-purpose device (e.g., a smartphone, a tablet, a laptop computer, or a desktop computer) in which an application or program for accessing a platform is installed or an access terminal dedicated to a platform.

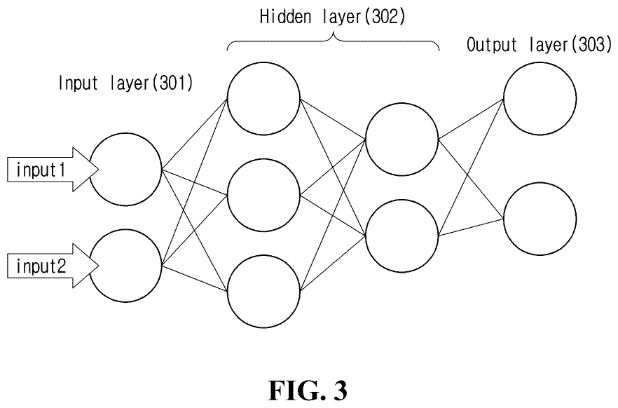

The server 120 provides a platform according to embodiments of the present disclosure. The server 120 may provide various functions for an off-label indication provision platform and operate an artificial intelligence (AI) model. An example of an artificial neural network applicable to the present disclosure will be described with reference to FIG. 3 below. In addition, the server 120 may perform learning for the AI model using learning data. According to various embodiments of the present disclosure, the server 120 stores a plurality of AI models for tasks of providing an off-label indication and optionally selects and uses at least one of the AI models. Here, the server 120 may be a local server present in a local network or a remote access server (e.g., a cloud server) that is connected via an external network. The server 120 may include a communication unit for communication, a storage unit that stores data and a program, and a processor for control.

FIG. 2 illustrates a structure of an apparatus according to an embodiment of the present disclosure. An apparatus 200 is a functional unit configured to use a common memory space for data necessary for the execution of a program. The apparatus 200 may be composed of at least one computer and software associated with the computer. The apparatus 200 may be understood as the structure of the server 120 of FIG. 1.

Referring to FIG. 2, the apparatus 200 may include a processor 201 that performs communication through a bus 207, a communication device 202, a memory 203, a storage device 204, and at least one of an input interface device 205 and an output interface device 206.

The processor 201 is hardware that serves to process various types of information in the apparatus 200. The processor 201 may be a central processing unit (CPU) or a semiconductor device that executes an instruction stored in the memory 203 and/or the storage device 204.

The communication device 202 is a data transmission device for exchanging data with another device or system in data communication. The communication device 202 may include a data input device or a communication control device. For example, the communication device 202 enables communication of voice, image, and text data between a data system and other devices.

The memory 203 is a memory device that is capable of storing information. The information includes a program or software required for the operation of the apparatus 200 and data that is generated during the operation. The memory 203 may include a read only memory (ROM) and a random access memory (RAM). Here, the RAM may load data and store modified content again after processing a required task. The ROM is a read-only memory device, and data stored in the ROM may be preserved permanently or semi-permanently.

The storage device 204 may store various types of information that is processed in the apparatus 200. The storage device 204 may include various forms of volatile or non-volatile storage media.

The input interface device 205 may detect a command from a user, enabling the user to operate the system. In addition, the output interface device 206 may display a use result of a user in the system. The input interface device 205 and the output interface device 206 may be a user interface (UI).

A method or a step of an algorithm described in relation to the embodiments described in the present specification may be directly implemented as hardware and a software module that are executed by the processor 201 or a combination thereof. The software module may reside in a storage medium (i.e., the memory 203 and/or the storage device 204) such as a RAM, a flash memory, a ROM, an erasable programmable read-only memory (EPROM), an electrically erasable programmable read-only memory (EEPROM), a register, a hard disk, a removable disk, and a CD-ROM.

An exemplary storage medium is coupled to the processor 201, and the processor 201 may read information from the storage medium and write information in the storage medium. As another example, the storage medium may be integrated with the processor 201. The processor 201 and the storage medium may reside in an application specific integrated circuit (ASIC). The ASIC may reside in a user terminal. As another method, the processor and the storage medium may reside in the user terminal as individual components.

FIG. 3 illustrates a structure of an artificial neural network applicable to a system according to an embodiment of the present disclosure. The artificial neural network shown in FIG. 3 may be understood as a structure of AI models stored in the server 120 or a third device that is capable of operating in conjunction with the server 120. In addition, the artificial neural network shown in FIG. 3 may be understood as a structure of models used in the present disclosure, and may also be understood as a structure of a feed forward neural network (FFNN) in a model.

Referring to FIG. 3, the artificial neural network consists of an input layer 301, at least one hidden layer 302, and an output layer 303. Each of the layers 301, 302 and 303 consists of a plurality of nodes, and each of the nodes is connected to an output of at least one node that belongs to a previous layer. Each node calculates the inner product of each output value from nodes of a previous layer and a corresponding connection weight and then transmits an output value obtained by multiplying the inner product by a non-linear activation function to at least one neuron of a next layer.

The artificial neural network shown in FIG. 3 may be formed by learning (e.g., machine learning or deep learning). In addition, an artificial neural network model used in various embodiments of the present disclosure may include at least one of a fully convolutional neural network, a convolutional neural network, a recurrent neural network, a restricted Boltzmann machine (RBM), and a deep belief neural network (DBN) but is not limited thereto. Alternatively, a machine learning method other than deep learning may be included. Alternatively, a hybrid-type model that combines deep learning and machine learning may be included. For example, a feature of an image may be extracted by applying a deep learning-based model, and an image may be classified or recognized based on the extracted feature by applying a machine learning-based model. The machine learning-based model may include a support vector machine (SVM) and AdaBoost but is not limited thereto.

The present disclosure is directed to utilizing an artificial intelligence to automatically detect and analyze an off-label use case in real world data of a medical organization. In an actual medical environment, there is a problem that a specific drug, despite showing excellent efficacy for a particular disease, does not obtain regulatory approval and thus lacks legal protection due to the high cost and time required for clinical trials. In addition, when a drug for treating a specific disease includes an adjunctive drug, there is a problem that the disease is classified as an indication for the adjunctive drug, even though the adjunctive drug is unrelated to the disease.

To solve the above-described problems, the present disclosure may provide the latest treatment trends and off-label indications by utilizing AI technology based on real world data (RWD). Specifically, the present disclosure may extract and standardize healthcare data and approval information of active ingredients, which are collected from actual medical environment, by utilizing AI technology, thereby collecting indication-related data. Based on the collected data, a relationship between an active ingredient and an indication may be analyzed using a graph database (GraphDB). In addition, the analysis of the relationship between the active ingredient and the indication makes it possible to utilize the scope of the active ingredient's actual and predicted indications. Through such a process, the present disclosure may provide services to various users, including healthcare professionals and patients seeking current therapeutic trends, a drug utilization review (DUR) system requiring information on new indications and management of issues related to off-label use, a regulatory body conducting routine safety evaluations for pharmacovigilance, and various pharmaceutical or medical organizations.

To provide an off-label indication, the present disclosure may use an LLM-based AI, a system for mapping extracted terms to an internationally standardized vocabulary for analysis, a KnowledgeDB for data visualization, and a mining technique for relationships within the data.

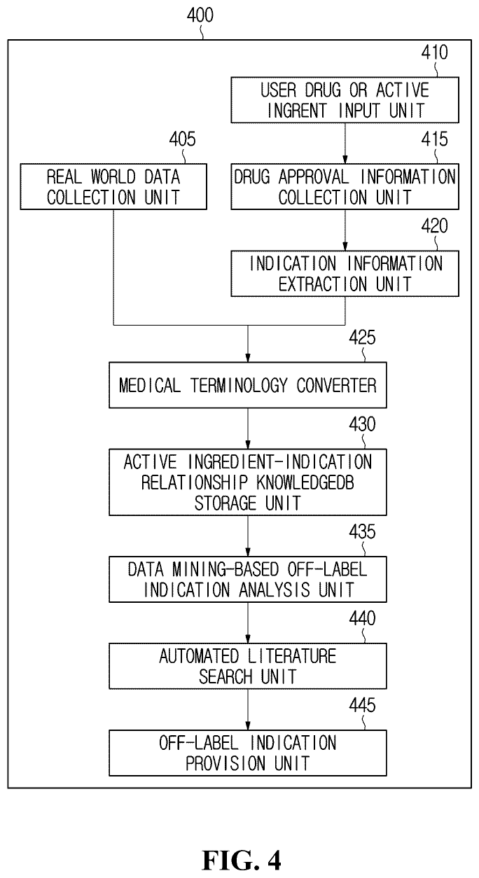

FIG. 4 illustrates a structure of an apparatus that provides an off-label indication according to an embodiment of the present disclosure. Referring to FIG. 4, an off-label indication provision apparatus 400 may include a real world data collection unit 405, a user drug or active ingredient input unit 410, a drug approval information collection unit 415, an indication information extraction unit 420, a medical terminology converter 425, an active ingredient-indication relationship knowledgeDB storage unit 430, a data mining-based off-label indication analysis unit 435, an automated literature search unit 440, and an off-label indication provision unit 445.

The real world data collection unit 405 may collect an indication for an active ingredient. Specifically, the real world data collection unit 405 may query a list of usage volumes according to each active ingredient and/or disease through a drug use information query service for healthcare big data. The real world data collection unit 405 may collect an indication for an active ingredient based on the found list of usage volumes. Here, the indication may be a disease or symptom for which a particular drug or surgery is considered likely to be effective. In addition, the term “indication” may be used interchangeably with “medical indication”.

The user drug or active ingredient input unit 410 may receive a drug and/or active ingredient that a user wants to analyze. Here, the drug and/or active ingredient to be analyzed may be directly input by the user. The drug approval information collection unit 415 may collect active ingredient approval information using the active ingredient approval information API that is provided by the Ministry of Food and Drug Safety. Specifically, the drug approval information collection unit 415 may use the active ingredient approval information API to collect approval information related to a drug and/or active ingredient that is input from the user drug or active ingredient input unit 410.

The indication information extraction unit 420 may extract disease and/or symptom information including an indication from the approval information that is collected by the drug approval information collection unit 415. Specifically, the indication information extraction unit 420 may extract information on an indication, a disease, and/or a symptom from the approval information collected by the drug approval information collection unit 415 by using an LLM prompt. Here, the LLM prompt is an input provided by a user or system and may be an instruction that requests an LLM to perform a specific task. The prompt may have various forms such as a question, a command, and the beginning of a sentence.

The LLM, that is, a large language model, may be an AI system capable of grasping and generating language from a huge amount of text data. This LLM may generate an appropriate response based on the prompt. As an example, the user or system may request the LLM to extract information on an indication, a disease, and/or a symptom from approval information of a specific drug. In response to the request, the LLM may generate an appropriate response to the requested information from the user or system.

The medical terminology converter 425 may convert information on a disease and/or a symptom expressed in various languages into medical terms. Here, the medical terms may be MedDRA terms that are international standard terms, but the present disclosure is not limited thereto and may include all medical terms used in the art. The medical terminology converter 425 may utilize various embedding vector-based coding techniques to information on a disease and/or a symptom expressed in various languages into international standard terms.

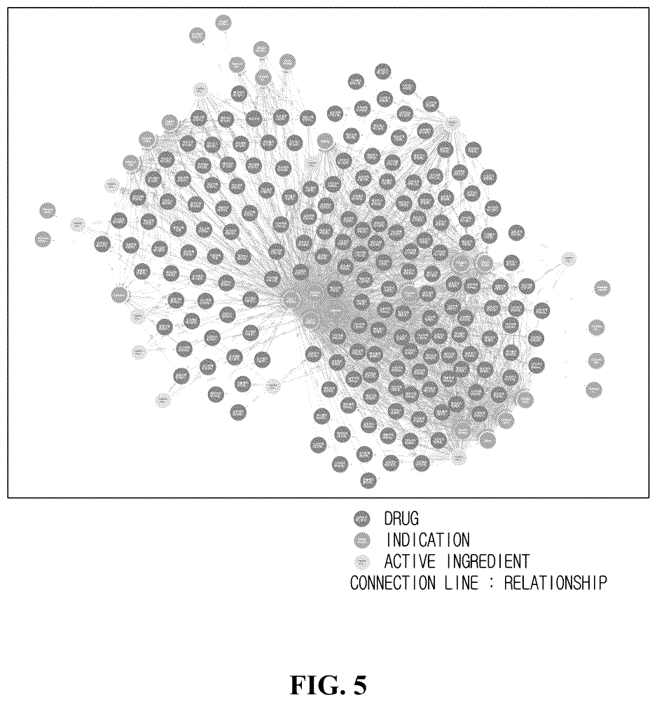

The active ingredient-indication relationship knowledgeDB storage unit 430 may generate and then store a constructed knowledgeDB that represents a relationship between an active ingredient and indication information. Here, the knowledgeDB may be relationship information and include, for example, a graphDB (i.e., graph information). As illustrated in FIG. 5, the graphDB may be a graph that represents a relationship between an active ingredient and a MedDRA term or a medical term used in the art for each of an indication extracted from approval information of the active ingredient and an indication collected from healthcare big data. However, the present disclosure is not limited thereto, and the knowledgeDB may include various forms of knowledge information that represent a relationship between an active ingredient and an indication. Here, the stored knowledgeDB may be used to analyze a relationship between an active ingredient and an indication. That is, the present disclosure may visualize a relationship between an active ingredient and indication information using a knowledgeDB, thereby enhancing relationship analysis.

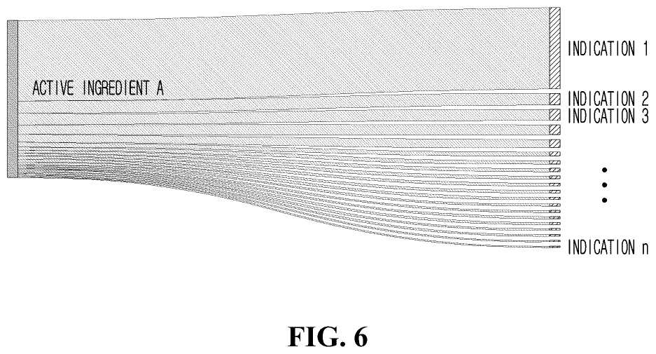

In addition, according to the present disclosure, as shown in FIG. 6, relationship information showing an indication not included in drug approval information among indications of a drug may be generated based on real world data and then may be stored. That is, relationship information may be generated using only indications not included in drug approval information among indications of a specific drug, and thus a relationship between the specific drug and an off-label indication may be visualized. According to an embodiment of the present disclosure, as shown in FIG. 6, off-label indications for an active ingredient A may be listed in descending order of use frequency or therapeutic effect (an indication 1, an indication 2, an indication 3, . . . , an indication n), and thus a user may easily identify an off-label indication associated with the active ingredient A. FIG. 6 describes an example of a relationship between an active ingredient and an indication, but the present disclosure is not limited thereto, and relationship information may also be generated based on a relationship between a drug and an indication.

The data mining-based off-label indication analysis unit 435 may exclude an indication with low relevance to an active ingredient. When medications are prescribed in an actual medical environment, not only a drug for treating a target disease but also an adjunctive drug may be prescribed under the same indication. Alternatively, when various therapeutic agents for multiple diseases are prescribed concurrently, they may be issued on a single prescription. In this case, an error may occur because of the lack of a clear match between each drug and a disease that the drug is used to treat. To improve this problem, the data mining-based off-label indication analysis unit 435 may use an associated rules technique to exclude an indication with low relevance to an active ingredient. That is, the data mining-based off-label indication analysis unit 435 may exclude an indication with low relevance to an active ingredient and thus filter off-label use of the active ingredient.

Here, the association rules technique is a data mining technique and may be a technique that is used to find a relationship between items in data. Specifically, the association rules technique may generate an association rule using an algorithm such as an Apriori algorithm and an FP-Growth algorithm. In this process, key metrics such as support, confidence, and lift may be calculated. Support may be a proportion represented by a specific itemset in total data. Confidence may be a probability that a certain indication will appear when a specific drug is present. Lift may be a metric of how much more likely the actual association between a specific drug and an indication is than random chance. Accordingly, the association rules technique interprets an association rule using support, confidence, and lift and thus may evaluate the possibility that a specific drug will be used for a specific indication.

The automated literature search unit 440 may automatically search for literature related to a drug and a new indication to verify a found indication. Specifically, the automated literature search unit 440 may automatically search for literature related to a drug and a corresponding indication to verify information filtered by the data mining-based off-label indication analysis unit 435.

The off-label indication provision unit 445 may suggest an off-label indication. Specifically, the off-label indication provision unit 445 may suggest an off-label indication by analyzing the possibility of using a drug based on an indication provided through data mining and relevant literature.

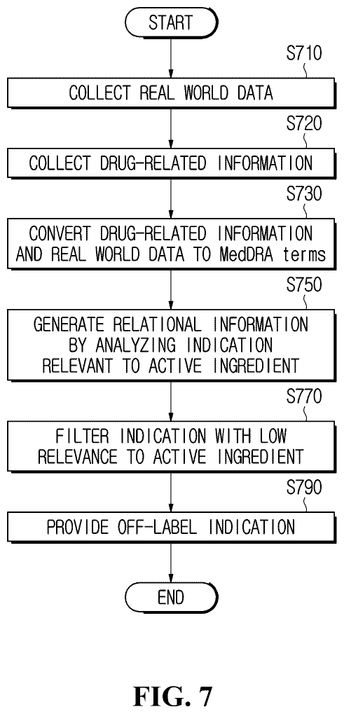

FIG. 7 is a flowchart of a method of providing an off-label indication according to an embodiment of the present disclosure. Referring to FIG. 7, real world data may be collected (S710). As an example, an off-label indication provision system (hereinafter referred to as “indication system”) may collect real world data from healthcare big data.

In addition, the indication system may collect drug-related information (S720). Here, the drug-related information may include at least one of drug information, approval information, or an indication. Specifically, the indication system may receive information on an active ingredient of a drug from a user. The indication system may collect drug approval information based on the received information on the active ingredient of the drug and extract an indication based on the drug approval information. That is, the indication system may extract the approval information and/or the indication based on the information on the active ingredient of the drug that is input by the user.

Here, the indication system may use an LLM to extract the indication. That is, the indication corresponding to the information on the active ingredient of the drug that is input by the user may be extracted based on the LLM. For example, when the user inputs an active ingredient A into the indication system, the indication system may collect approval information of the active ingredient A and extract an indication of the active ingredient A based on the collected approval information. Here, in order to collect the approval information of the drug, the indication system may use the active ingredient approval information API provided by the Ministry of Food and Drug Safety.

Next, the indication system may convert the drug-related information and the real world data into international standard terms (S730). Here, the international standard terms may be MedDRA terms, but the present disclosure is not limited thereto and may include various medical terms used in the art. The indication system may analyze an indication associated with the active ingredient of the drug to generate relationship information (S750). Specifically, the indication system may store every indication associated with the active ingredient of the drug input by the user as the relationship information. Here, the relationship information may include graph information as shown in FIG. 5 and/or FIG. 6. However, the present disclosure is not limited thereto, and various types of relationship information may be generated and then stored.

The indication system may filter an indication with low relevance to the active ingredient of the drug (S770). That is, the indication system may perform filtering by removing information on an indication with low relevance to the active ingredient of the drug from all indication information that is analyzed at S750. Here, the filtering may be performed based on association rules data mining, and the association rules data mining may be performed in further consideration of the real world data. Thus, the indication system may provide a more accurate off-label indication by filtering the information on the indication with low relevance (S790).

According to an embodiment, the indication system may automatically search for literature relevant to the drug-related information. In addition, the indication system may verify the filtered relationship information based on the searched literature. That is, the indication system may check the filtered final result again in consideration of the searched literature, thereby improving the accuracy of a provided off-label indication.

Thus, the present disclosure may utilize an AI to automatically detect and analyze an off-label use case in real world data of a medical organization. Accordingly, a user may use the indication system as supporting evidence for drug repositioning. In addition, a pharmaceutical company may use the indication system to monitor off-label use and explore the possibility of expanding an indication.

In addition, a healthcare professional may use the indication system to identify the latest drug treatment trends. In addition, the Health Insurance Review and Assessment Service (HIRA) may use the indication system for the evaluation of prescribing appropriateness, thereby considering the redistribution of medical resources. In addition, a regulatory agency may also use the indication system as an opportunity for data-driven healthcare by identifying potential risk factors.

Although the exemplary methods of the present disclosure are presented as a series of operations for clarity of explanation, this is not intended to limit a sequence in which the operations are performed, and the respective operations may be performed simultaneously or in a different order, when necessary. To implement a method according to the present disclosure, other operations may be included in addition to the exemplified operations, or some operations may be excluded and additional other operations may be included.

The various embodiments of the present disclosure are not an exhaustive list of all possible combinations but are intended to describe representative aspects of the invention, and the elements described in the various embodiments may be applied independently or in a combination of two or more.

In addition, the various embodiments of the present disclosure may be implemented using hardware, firmware, software, or a combination thereof. In the case of a hardware implementation, it may be implemented by one or more of an application specific integrated circuit (ASIC), a digital signal processor (DSP), a digital signal processing device (DSPD), a programmable logic device (PLD), a field programmable gate array (FPGA), a general processor, a controller, a microcontroller, or a microprocessor.

The scope of the present disclosure includes software or machine-executable instructions (e.g., an operating system, an application, firmware, or a program) that enable the operations of the method according to the various embodiments to be executed on an apparatus or a computer and a non-transitory computer-readable medium that stores such software or instructions to be executable on the apparatus or the computer.

What is claimed is:

1. A method for providing an off-label indication in an off-label indication provision system, the method comprising:
collecting real world data from health care big data;
collecting drug-related information;
converting the drug-related information and the real world data to international standard terms;
generating relationship information by analyzing an indication relevant to an active ingredient based on the drug-related information and the real world data that are converted to the international standard terms;
filtering an indication with low relevance to the active ingredient from the relationship information; and
providing an off-label indication based on the filtered relationship information.

2. The method of claim 1, wherein the collecting of the drug-related information further includes:
receiving drug information from a user;
collecting drug approval information based on the drug information; and
extracting an indication based on the drug approval information.

3. The method of claim 2, wherein the indication is extracted based on a large language model (LLM).

4. The method of claim 2, wherein the drug approval information is collected based on an active ingredient approval information application programming interface (API) that is provided by the Ministry of Food and Drug Safety.

5. The method of claim 2, wherein the filtering is performed based on association rules data mining.

6. The method of claim 5, wherein the association rules data mining is performed further based on the real world data.

7. The method of claim 5, wherein the association rules data mining is performed based on at least one of support, confidence, or lift.

8. The method of claim 1, further comprising:
automatically searching for literature relevant to the drug-related information; and
verifying the filtered relationship information based on the searched literature,
wherein the provision of the off-label indication is performed based on the verification.

9. An apparatus for providing an off-label indication in an off-label indication provision system, the apparatus comprising:
a storage unit that stores necessary information for operating the apparatus; and
a processor coupled to the storage unit,
wherein the processor is configured to:
collect real world data from health care big data,
collect drug-related information,
convert the drug-related information and the real world data to international standard terms,
generate relationship information by analyzing an indication relevant to an active ingredient based on the drug-related information and the real world data that are converted to the international standard terms,
filter an indication with low relevance to the active ingredient from the relationship information, and
provide an off-label indication based on the filtered relationship information.

10. The apparatus of claim 9, wherein the processor is further configured to:
receive drug information from a user,
collect drug approval information based on the drug information, and
extract an indication based on the drug approval information.

11. The apparatus of claim 10, wherein the indication is extracted based on a large language model (LLM).

12. The apparatus of claim 10, wherein the drug approval information is collected based on an active ingredient approval information application programming interface (API) that is provided by the Ministry of Food and Drug Safety.

13. The apparatus of claim 10, wherein the filtering is performed based on association rules data mining.

14. The apparatus of claim 13, wherein the association rules data mining is performed further based on the real world data, and
the association rules data mining is performed based on at least one of support, confidence, or lift.

15. The apparatus of claim 9, wherein the processor is further configured to:
automatically search for literature relevant to the drug-related information, and
verify the filtered relationship information based on the searched literature, and
wherein the provision of the off-label indication is performed based on the verification.
